(19) United States
(12) Patent Application Publication
(10) Pub. No.: US 2002/0168760 A1
(43) Pub. Date: Nov. 14, 2002

(54) Retroviral vectors for gene transfer into neuronal cells

(76) Inventors: Ralph C. Dornburg, Philadelphia, PA (US); Matthias J. Schnell, Harleysville, PA (US); Bernhard Dietzschold, Newtown Square, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 10/097,556

(22) Filed: Mar. 13, 2002

Related U.S. Application Data
(60) Provisional application No. 60/275,244, filed on Mar. 13, 2001.

(51) Int. Cl. C12Q 1/70; A61K 39/12; A61K 39/21; C12N 15/00; C12N 15/09; C12N 15/63; C12N 15/70; C12N 15/74

(52) U.S. Cl. 435/320.1; 435/5; 424/199.1; 424/204.1; 424/207.1

(57) ABSTRACT

The current methods of gene therapy do not allow for the efficient transduction of nerve cells, thereby limiting treatment of diseases or disorders involving the nervous system. The present invention is a method of treating a disease or disorder wherein an avian retrovirus (spleen necrosis virus, SNV) is engineered to express a rabies virus glycoprotein that allows for the specific targeting of nerve cells. Since SNV is not infectious to human cells the retrovirus of the present invention is safe. Further, incorporation of a glycoprotein gene, specifically the N2C gene, and a therapeutic gene(s) of interest into the retroviral vector allows for the specific and efficient transduction of nerve cells with the gene(s) of interest, thereby treating a disease or disorder involving nerve cells.

### CROSS REFERENCE TO RELATED APPLICATIONS

This application claims priority under 35 U.S.C. §119 based upon U.S. Provisional Patent Application No. 60/275,244, filed Mar. 13, 2001.

### GOVERNMENT RIGHTS IN THE INVENTION

This invention was made with government support under grant 1RO1 AI41899-01 awarded by the National Institute of Health. The government has certain rights in the invention.

### FIELD OF THE INVENTION

The present invention relates to the fields of molecular biology and gene therapy, and to a retroviral vector displaying a rabies virus glycoprotein gene for targeted delivery of a gene(s) of interest into neuronal cells and, more particularly, to a method of treating or preventing a neurological disease or condition.

### BACKGROUND OF THE INVENTION

Current methods for expressing an exogenous gene in a mammalian cell include the use of mammalian viral vectors, such as those that are derived from retroviruses, adenoviruses, herpes viruses, vaccinia viruses, polio viruses, or adeno-associated viruses. Other methods of expressing an exogenous gene in a mammalian cell include direct injection of DNA, the use of ligand-DNA conjugates, the use of adenovirus-ligand-DNA conjugates, calcium phosphate precipitation, and methods that utilize a liposome- or polycation-DNA complex.

Typically, viruses that are used to express desired genes are constructed by removing unwanted characteristics from a virus that is known to infect, and replicate in, a mammalian cell. For example, the genes encoding viral structural proteins and proteins involved in viral replication often are removed to create a defective virus, and a therapeutic gene is then added. This principle has been used to create gene therapy vectors from many types of animal viruses such as retroviruses, adenoviruses, and herpes viruses. The virus vector of the present invention utilizes the spleen necrosis virus (SNV), a C-type retrovirus, an avian virus that does not normally infect humans. However, the avian viruses efficiently infect human cells and insert their genome into that of the host cell when they are pseudotyped with envelope proteins which display targeting ligands specific for human cell surface receptors.

Retroviruses are wide-spread in nature and are associated with various diseases in animals and man. (Varmus, H. E. & Brown, P., Retroviruses 53-108, 1998). They contain an RNA genome, which is converted into a double-stranded DNA copy. This DNA copy is inserted into the genome of the host cell. Thus, a retrovirus becomes part of the genomic outfit of the infected cell. Some retroviruses carry non-retroviral genes, which have been acquired from the infected cell by recombination. Thus, retroviruses act as natural gene transfer vectors. These features, as well as the high efficiency of this process, has led to the construction of retroviral vectors useful for the transfer of many non-retroviral genes into a large variety of different cells. (Dornburg, R., Gene Ther 2:301-310, 1995; Eglitis, M. A. & Anderson, W. F., Biotechniq 6:608-614, 1988; Miller, A. D., Hum. Gene. Ther. 1:5-14, 1990). In recent years, retroviral vectors have also been used to transfer therapeutic genes into human cells to treat genetic diseases, cancer, AIDS, and various other diseases. (Anderson, W. F., Nature 392:25-30, 1998).

All current retroviral vector systems consist of two components: the retroviral vector, which is a genetically modified viral genome containing the gene of interest replacing retroviral protein coding sequences, and a helper cell that supplies the retroviral proteins for the encapsidation of the vector genome into retroviral particles (FIG. 1 shows a spleen necrosis virus (SNV, a C-type retrovirus) vector system as an example). Modern helper cells contain separate plasmid constructs which express all retroviral proteins necessary for replication (FIG. 1). After transfection of the vector genome into such helper cells, the vector genome is encapsidated into virus particles, due to the presence of specific encapsidation sequences. Virus particles are released from the helper cell carrying a genome containing only the gene(s) of interest (FIG. 1). Thus, once established, retrovirus helper cells can produce gene transfer particles for very long periods of time (e.g., several years). In the last decade, several retroviral vector systems have also been derived from other C-type retroviruses. (Dornburg, R., Gene Ther 2:301-310, 1995; Gunzburg, W. H. & Salmons, B., Journal of Molecular Medicin-Jmm 74:171-182, 1996; Miller, A. D., Hum. Gene. Ther. 1:5-14, 1990).

All retroviral vectors currently used or aimed to be used in human gene therapy have several shortcomings. First, they have a very broad host range. For example, retroviral vectors derived from murine leukemia virus infect a large variety of cell types in many mammalian species. These vectors cannot infect quiescent cells. Retroviral vectors derived from lentiviruses and pseudotyped with the envelope protein of vesicular stomatitis virus (VSV) are able to infect quiescent cells but also infect numerous cell types of almost all species tested. While HIV-1 derived vectors pseudotyped with the VSV envelope can infect neurons, the efficacy is very low. (Bukrinsky, M. I., et al., Nature 365:666-670, 1993; Lewis, P. & Emerman, M., J. Virol 68:510-516, 1994; Naldini, L., et al., Science 272:263-267, 1996; Schwedler, U., et al., Proc. Natl. Acad. Sci. USA 91:6992-6996, 1999). Thus, vectors of the current state of art lack specificity for a particular cell type, such as neuronal cells. The present invention overcomes this by using a retroviral (SNV) based vector system to express a glycoprotein gene, specifically the N2C G protein of a rabies virus, to target delivery of a gene(s) of interest into neuronal cells.

Rabies viruses (RVs) are highly neurotropic viruses that usually cause a fatal infection in all warm-blooded species, with virus replication primarily occurring in neurons. RVs are non-segmented, negative-strand RNA viruses. The viral genome encodes five structural proteins including a nucleoprotein (N), phosphoprotein (P), matrix protein (M), RNA-dependent RNA polymerase (L), and a transmembrane glycoprotein (envelope). The single transmembrane envelope protein is responsible for both the attachment to the host cell's receptor and the fusion and release of the viral core into the cytoplasm. Of note, different RV strains differ greatly in their preference to infect neuronal cells. Two variant virus strains, CVS-N2c and CVS-B2c that differ genotypically and phenotypically were isolated from the mouse-adapted rabies virus strain CVS-24 (challenge virus standard). The envelope protein of the strain CSV-N2c is highly specific for neuronal cells and has a low affinity to other cell types, whereas the envelope protein of the rabies virus strain CVS-B2c does not have a preference for neuronal cells. (Dietzschold, B., et al., Journal of Human Virology January-February 3:50-57, 2000; Morimoto, K., et al., Proceedings of the National Academy of Sciences of the United States of America 95:3152-3156, 1998; Morimoto, K., et al., Journal of Virology 73:510-518, 1999). In addition, the envelope protein from the RV-vaccine strain SAD B19 does infect neuronal cells and other cells lines at the same level.

The present invention relates to genetically engineered retroviral vector particles capable of specifically transducing genes into neuronal cells. The genetically engineered retroviral vector particle contains retroviral core proteins or genetically engineered core proteins, a transducing gene(s) of interest, and the envelope protein of a rabies virus (FIGS. 2 and 3).

All retroviral vectors currently used in human gene therapy trials contain the envelope protein of amphotropic (ampho) murine leukemia virus (MLV) or that of VSV. Ampho-MLV, as well as VSV, has a very broad host range and can infect various tissues of many species including humans. (Eglitis, M. A. & Anderson, W. F., Biotechniq 6:608-614, 1988; Miller, A. D., Hum. Gene. Ther. 1:5-14, 1990). Thus, the use of vectors containing such envelope proteins enables the transduction into many different human tissues. However, due to this broad host range, gene transfer has to be performed ex vivo. If injected directly into the blood stream, the chances that the vector particles will infect their actual target cells is very low. Furthermore, such vector particles may infect germ line cells, which are continuously dividing. Thus, the target cells have to be isolated and the gene transfer performed in tissue culture. Gene transduced cells are then selected and re-introduced into the patient.

This method of gene transduction has major shortcomings in regard to human gene therapy. First, it is very expensive and requires highly-trained personnel. Second, human cells that are kept in tissue culture change their physiological behavior and/or take up fetal bovine proteins (a component of the tissue culture medium) and display bovine peptides via the human leukocyte antigen system (HLA) on the cell surface. Consequently, such cells become immunogenic and are eliminated by the immune system of the patient. In addition, several cell-types, most particularly neuronal cells, cannot be cultivated and expanded in vitro. To bypass such ex vivo protocols, efforts are now underway in many laboratories to develop cell-type-specific gene delivery systems, which would enable the injection of the gene delivery vehicle directly into the patient's blood stream or tissue of interest.

The cell-type specificity of a virus particle is determined by the nature of the retroviral envelope protein which mediates the binding of the virus to a receptor of the target cell. (Hunter, E. & Swanstrom, R., Immunol. 157:187-253, 1990). Thus, experiments have been initiated in several laboratories to modify the envelope protein of retroviruses in order to alter the host range of the vector. The reticuloendotheliosis viruses strain A (REV-A) and SNV are avian retroviruses, which are not naturally infectious in human cells. For example, retroviral vector particles derived from SNV have been developed which display the antigen binding site of an antibody (single chain antibodies, scAs) on the viral surface. Particles that displayed various scAs against human cell surface proteins were competent for infection into human cells that express the antigen recognized by the antibody. (Chu, T. H. & Dornburg, R., J. Virol. 69:2659-2663, 1995; Chu, T. H. & Dornburg, R., J. Virol. 71:720-725, 1997; Chu, T. H., et al., Gene. Ther. 1:292-299, 1994; Jiang, A., et al., J. Virol 72:10148-10156, 1998). The infectivity was mediated by the scA. Moreover, parts of the envelope protein of the human immunodeficiency virus (HIV-1) displayed on SNV-derived vectors enabled the efficient infection of CD4+ cells, the primary target cell of HIV-1. (Jiang, A., et al., Human. Gene Therapy 10:2627-2636, 1999).

The present invention fulfills a long sought, yet unfulfilled, need for gene therapy targeted specifically to neuronal cells. A major obstacle in the current retroviral vector systems is that the currently available vectors are incapable of specifically infecting neuronal cells. The retroviral vectors of the present invention overcome this by displaying the envelope protein (glycoprotein) of a rabies virus, thereby allowing for the highly efficient infection of neuronal cells. Vectors pseudotyped with the envelope of the rabies virus strain N2C enable cell-type-specific gene delivery into neuronal cells.

### SUMMARY OF THE INVENTION

It is an object of the present invention to generate a retroviral vector particle with retroviral core proteins, a vector wherein the genome comprises an exogenous gene(s) of interest, a mammalian active promoter operably linked to the exogenous gene(s) of interest, and wherein the retroviral vector particle displays an envelope protein of a rabies virus. In one embodiment the retroviral vector particle has a mammalian active promoter that is a cell-type-specific promoter. In another embodiment the mammalian active promoter is an inducible promoter. In a further embodiment the retroviral vector particle core proteins are derived from at least one of a spleen necrosis virus (SNV) or a reticuloendotheliosis virus (REV-A). In another embodiment the retroviral vector particle envelope protein is a glycoprotein of a rabies virus N2C strain.

Another object of the invention is to present a method for treating a disease or disorder in a mammal. A therapeutically effective amount of a retrovirus vector encoding an exogenous gene(s) of interest within a retrovirus vector particle is administered to a mammal. The retrovirus vector particle is pseudotyped with an envelope protein of a rabies virus so that the target cell is transduced with the exogenous gene(s) of interest and the exogenous gene(s) of interest is subsequently expressed in the mammal. In one embodiment the envelope protein is an N2C glycoprotein. In another embodiment the target cell is a nerve cell.

### DESCRIPTION OF THE FIGURES

FIG. 1. The principle of a retroviral packaging line derived from a C-type retrovirus.

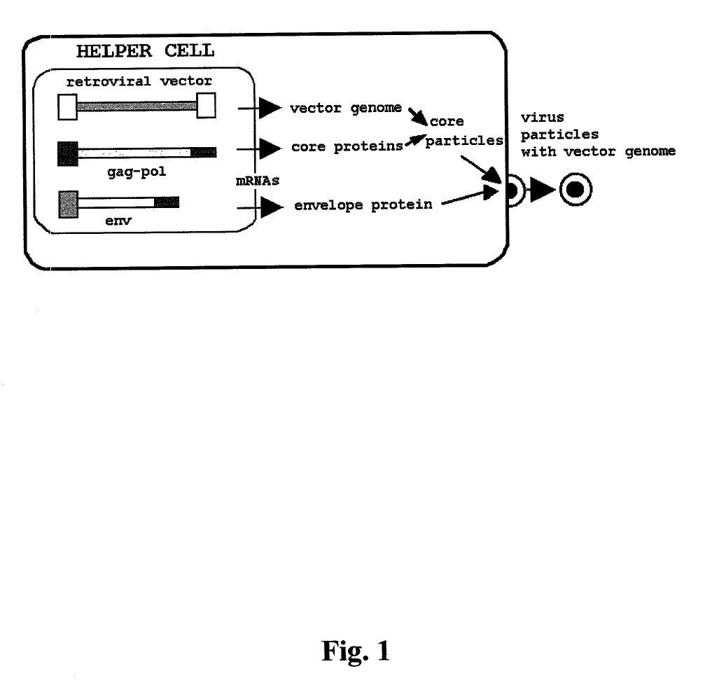

In helper cells retroviral proteins are expressed from different plasmid DNAs. These RNA transcripts do not contain encapsidation sequences. Thus, they are not encapsidated into retroviral particles. The RNA transcript of the retroviral vector contains an encapsidation sequence, and, therefore, is encapsidated into virions supplied by the helper cell. Supernatant tissue culture medium is used to infect fresh target cells. pro1 and pro2: promoters to express viral protein coding sequences; poly(A) polyadenylation sequence.

FIG. 2. Plasmid DNA constructs to generate retroviral packaging cells. pRD136 is a plasmid expressing spleen necrosis virus (SNV) gag-pol proteins and has been described previously. (Martinez, I. & Dornburg, R., Virology 208:234-241, 1995). Gag-pol is expressed from the murine leukemia virus (MLV) U3 promoter followed by the adenovirus tripartite leader sequence (AVtl) for enhanced gene expression. pZP33 is similar to pRD136 and expresses a chimeric gag-pol protein from the cytomegalovirus promoter. Gag proteins have been derived from the reticuloendotheliosis virus strain A (REV-A), the pol gene is that from SNV. To enable infection of quiescent cells, a nuclear translocalization sequence has been introduced into matrix protein sequence (MA) of Gag as described recently. (Parveen, Z., et al., Nature Biotechnology 18:623-629, 2000). The chimeric gag-pol gene is expressed from the cytomegalovirus immediate early promoter (CMV-pro). pZP36 is a plasmid containg a SNV-derived retroviral vector transducing the bacterial beta-galactosidase gene. However, the U3 promoters in both long terminal repeats (LTRs) are replaced with the immediate early promoter of cytomegalovirus (CMV). Plasmids pB2C and pN2C express envelope proteins of the rabies virus strains B2C and N2C, respectively.

FIG. 3. The components of a retroviral packaging line capable of infecting neuronal cells. 293T cells or other eucaryotic cell lines are transfected with 3 plasmids which contain (i) a retroviral vector genome containing the gene(s) of interest, (ii) a gene unit including promoter and polyadenylation signal sequences for expression of retroviral core proteins, and (iii) a plasmid to express a rabies virus envelope protein. Cells transfected with these three plasmids produce retroviral vector particles, which display the rabies virus envelope on the viral surface and which transduce the gene(s) of interest into fresh target (e.g., neuronal) cells.

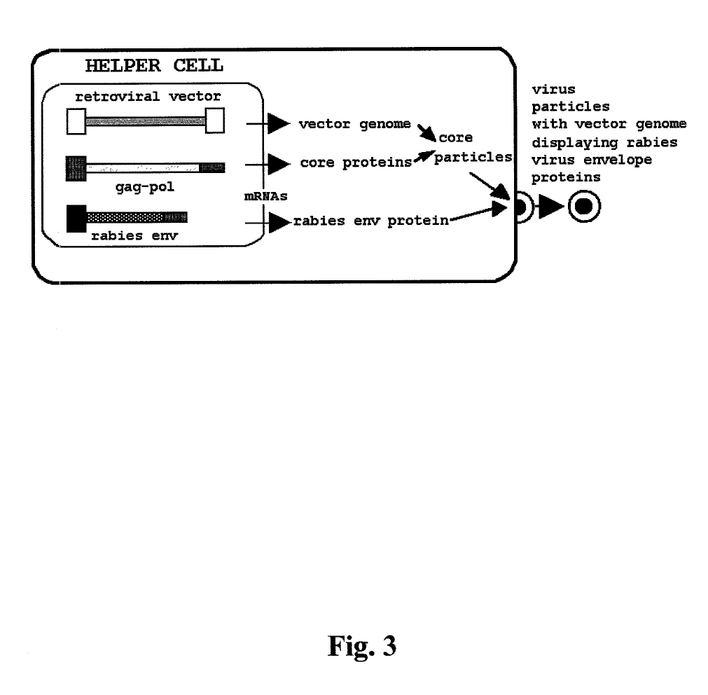

FIG. 4. Neuronal cells infected with retroviral vector particles which are pseudotyped with the envelope protein of a rabies virus. Cells are infected with retroviral vectors transducing the bacterial lacZ gene and pseudotyped with the envelope protein of the rabies virus strain N2C. Blue cells indicate successful gene transfer.

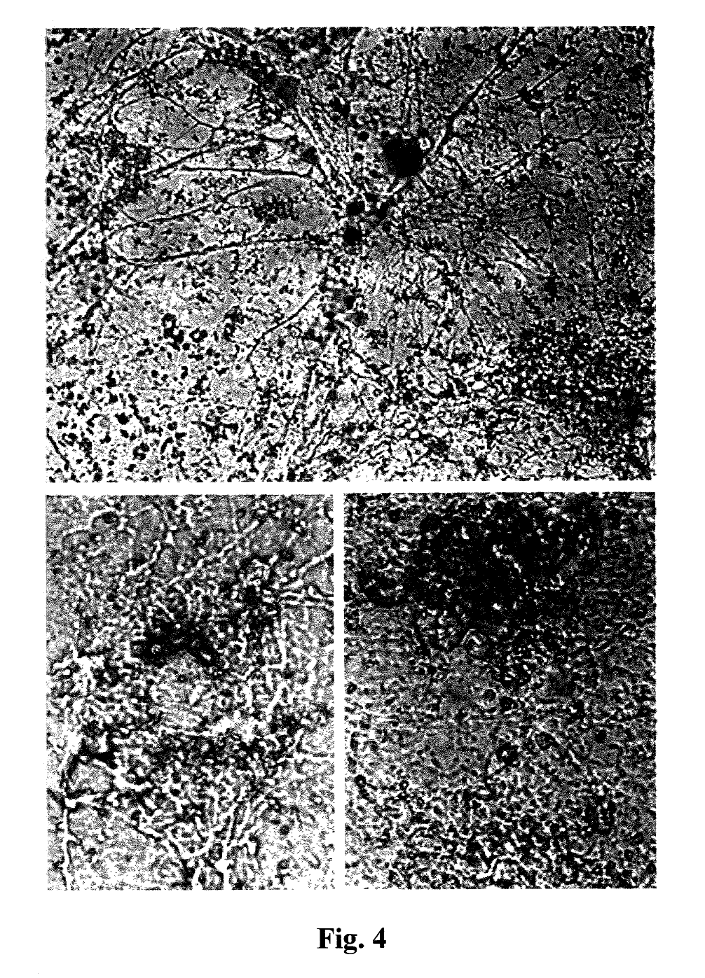

FIG. 5. Mouse brain cells infected in vivo. 2×10⁵ vector particles transducing the bacterial lacZ gene and pseudotyped with the envelope protein of the rabies virus strain N2C are injected into the right lateral brain lobe of newborn C57 BL/6 J-twi mice at day 0. Two months after the injection, the mice are sacrificed, the brain is removed, and stained with X-gal. A) photograph of the complete right lateral brain lobe of an infected mouse. B and C): photograph of thin sections of the right lateral lobe. Blue cells indicate successful gene transfer.

### DESCRIPTION OF THE INVENTION

Methods

Plasmid constructs. Plasmid DNAs are constructed following standard DNA cloning procedures. Retroviral core protein coding sequences or envelope genes are cloned into the universal eucaryotic gene expression vectors PRD114 or pWS4, which have been described in detail previously. (Sheay, W., et al., Biotechniq 15:856-861, 1993). Plasmid pRD136 expresses SNV gag-pol proteins and has been described previously. (Martinez, I. & Dornburg, R., Virology 208:234-241, 1995). Plasmid pZP33 is similar to pRD136. However, it contains the gag coding sequences of the reticuloendotheliosis virus strain A (REV-A). Using site-directed mutagenesis, a nuclear translocalization signal sequence is introduced into the matrix gene (MA) similar to plasmid construct pRD136-m7, which has been described in detail recently. (Parveen, Z., et al., Nature Biotechnology 18:623-629, 2000). Gag-pol are expressed from the cytomegalovirus (CMV) immediate early promoter. Plasmid pMD.G expresses the VSV envelope protein and has been described previously. (Naldini, L., et al., Science 272:263-267, 1996). Plasmid pIM29 expresses the SNV envelope protein and has been described previously. (Martinez, I. & Dornburg, R., Virology 208:234-241, 1995). Plasmids pN2C and pB2C are constructed in the following way: Polymerase chain reaction (PCR) is used to amplify the envelope coding sequences from cloned rabies virus isolates CVS-N2c and CVS-B2c, respectively. The primers used are 5′CCTCTAGAAGATGGTTCCTCAGGCTC3′ (SEQ. ID. NO: 1), which binds to the area incorporating the ATG initiation site and 5′TATAGGGCCCAAGCTTTCACAGTCTGATCTCACCTC3′ (SEQ. ID. NO: 2), which binds to the area incorporating the stop codon, thereby amplifying the protein coding sequences of N2C and B2C envelope protein. The PCR products are digested with XbaI and HindIII and the resulting DNA fragments are cloned into the universal gene expression vector pWS4 (Sheay, W., et al., Biotechniq 15:856-861, 1993) that is previously digested with XbaI and HindIII.

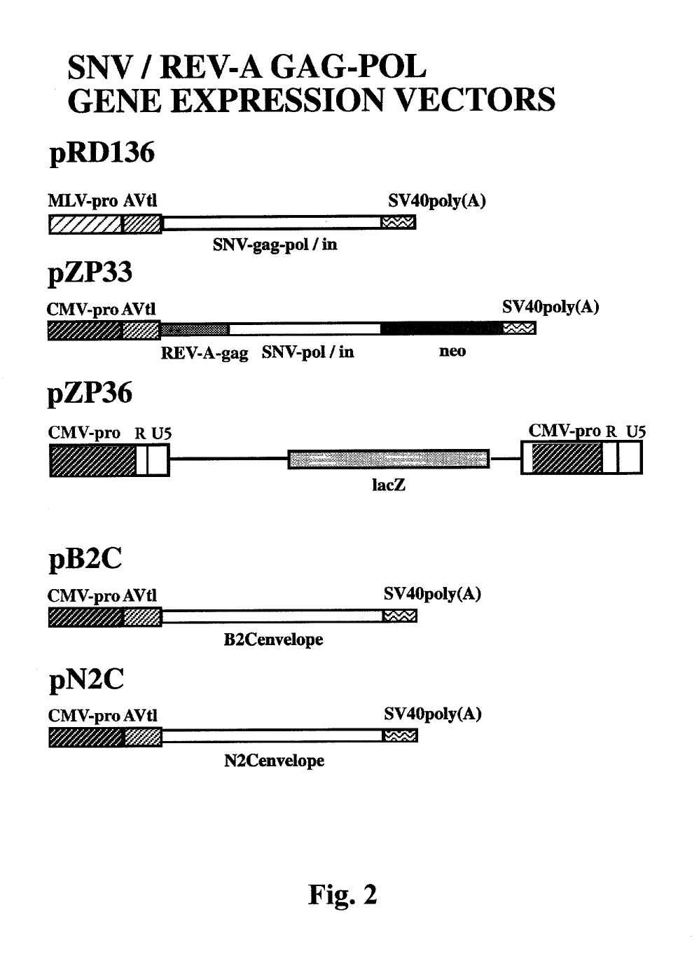

Generation of retroviral vector particles displaying rabies virus envelope proteins. To generate retroviral particles that display the envelope protein of a rabies virus, three different plasmids are transfected into human 293T cells. These plasmids are: (1) a plamid to express retroviral core proteins (e.g., pRD136 or pZP33, FIG. 2). (2) a plasmid containing a retroviral vector genome for the transduction of any exogenous gene(s) of interest, such as a marker gene (e.g., pZP36, FIG. 2), and (3) a plasmid expressing a rabies virus envelope protein (e.g., pB2C or pN2C, FIG. 2). The transfections are performed using the lipofectamin transfection protocol (supplied by Gibco) and following the procedure recommended by the supplier.

Infections to test retroviral vectors pseudotyped with rabies virus envelope proteins. To get a first insight into whether retroviral particles displaying rabies virus envelope proteins are infectious in neuronal cells, infection experiments are performed using tissue culture supernatant medium of 293T cells transfected with plasmids expressing retroviral core proteins, rabies virus envelope proteins, and a plasmid containing a retroviral vector genome containing the bacterial beta-galactosidase gene. 48 hours after transfection, the tissue culture supernatant of transfected cells is added to various target cells, such as dividing or growth-arrested neuronal or other cells (e.g., N2T cells, NA cells, BRS cells etc.). 48 hour after infection, the infected target cells are stained with X-gal as described previously. (Chu, T. H. & Dornburg, R. J. Virol. 69:2659-2663, 1995). This infection protocol has been used in earlier studies and has been described in detail in 22, herein incorporated by reference.

Growth-arrested neuronal cells. Methods have been established in several laboratories to growth-arrest cells at various stages of the cell-cycle. In our experiments, the protocol described in (Bukrinsky, M. I., et al., Nature 365:666-670, 1993; Lewis, P. & Emerman, M., J. Virol 68:510-516, 1994; Miyake, K., et al., Hum. Gene Ther. 9:467-475, 1999; Schwedler, U., et al., Proc. Natl. Acad. Sci. USA 91:6992-6996, 1999) and (Schwedler, U., et al., Proc. Natl. Acad. Sci. USA 91:6992-6996, 1999), is used, herein incorporated by reference.

Virus particle concentration. Virus particles harvested from transfected 293T cells (supra) are first filtered through a 22 μm filter and 30 ml of the virus solution is concentrated by ultracentrifugation in a Sorvall PC centrifuge at 23,000 rpm for 2 hours. The virus pellet is resuspended in 100 μl RPMI medium.

In vivo injections. 2 μl of concentrated virus solution (supra) is injected into the lateral lobe or the cerebellum of new-born mice (0 days old). The mice are placed on a mini light plate form and injections are performed using a magnifying glass of 1.7×3d (Scienceware) and a hamilton syringe.

Gene Therapy

Typically, the gene of interest is operably linked to a promoter that is active in the mammalian cell to be infected with the virus of the present invention. This mammalian-active promoter is used to drive expression of the exogenous gene of interest (e.g., a therapeutic gene). Where cell-type specific expression of the exogenous gene is desired, the exogenous gene in the genome of the virus is operably linked to a mammalian-active, cell-type-specific promoter, such as, but not limited to, a promoter that is specific for brain cells (e.g., neuronal cells), glial cells, or Schwann cells. For the expression of an exogenous gene specifically in neuronal cells, a neuron-specific enolase promoter can be used. (see Forss-Petter et al., Neuron 5:187-197, 1990). For expression of an exogenous gene in dopaminergic neurons, a tyrosine hydroxylase promoter can be used. For expression in pituitary cells, a pituitary-specific promoter such as POMC may be useful. (Hammer et al., Mol. Endocrinol. 4:1689-97, 1990).

Promoters that are inducible by external stimuli also can be used for driving expression of the exogenous gene. Such promoters provide a convenient means for controlling expression of the exogenous gene in a cell of a cell culture or within a mammal. Preferred inducible promoters include, but are not limited to, enkephalin promoters (e.g., the human enkephalin promoter), metallothionein promoters, mouse mammary tumor virus promoters, promoters based on progesterone receptor mutants, tetracycline-inducible promoters, rapamycin-inducible promoters, and ecdysone-inducible promoters. Methods for inducing gene expression from each of these promoters are known in the art.

The virus vector of the present invention can be introduced into the cell by administering the virus to a mammal that carries the cell. For example, the virus vector of the present invention can be administered to a mammal by subcutaneous, intravascular, or intraperitoneal injection. If desired, a slow-release device, such as an implantable pump, may be used to facilitate delivery of the virus to cells of the mammal. Additionally, transduction of motor neurons via retrograde transport after intramuscular injection will transduce sufficient numbers of cerebral, brainstem and/or spinal cord neurons. A particular cell type within the mammal can be targeted by modulating the amount of the virus vector of the present invention administered to the mammal and by controlling the method of delivery. For example, in a method of administration the virus is delivered to a tissue or organ containing the targeted cells of the mammal. Such administration can he accomplished by injecting a solution containing the virus vector of the present invention into a tissue, for example, but not limited to, the brain (e.g., the cerebral cortex). Alternatively, or in addition, administration can be accomplished by perfusing an organ with a solution containing the virus vector of the present invention, according to conventional perfusion protocols.

In another method, the virus vector of the present invention is administered intranasally, e.g., by applying a solution of the virus to the nasal mucosa of a mammal. This method of administration can be used to facilitate retrograde transportation of the virus vector into the brain. This method thus provides a means for delivering the virus vector to brain cells, (e.g., mitral and granule neuronal cells of the olfactory bulb) without subjecting the mammal to surgery. In an alternative method for using the virus vector of the present invention to express an exogenous gene in the brain, the virus vector is delivered to the brain by osmotic shock according to conventional methods for inducing osmotic shock. Additionally, administration of the viral vector of the present invention into the central nervous system can be intraventricular and intrathecal injection; intraventricular injection may further be facilitated by an intraventricular catheter, for example, attached to a reservoir, such as an Ommaya reservoir.

The amount of the viral vector of the present invention, which expresses a gene(s) of interest, which will be effective in the treatment of a particular disorder or condition will depend on the nature of the disorder or condition, and is determined by standard clinical techniques. In addition, in vitro assays may optionally be employed to help identify optimal dosage ranges. The precise dose to be employed in the pharmaceutical formulation (infra) will also depend on the route of administration, and the seriousness of the disease or disorder, and is decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

The invention can be used to express a variety of exogenous genes encoding gene products such as a polypeptides or proteins, expressing antisense RNAs, or expressing catalytic RNAs. Where the invention is used to express an antisense RNA, the antisense RNA can be complementary to a nucleic acid (e.g., an mRNA) of a pathogen of the mammalian cell (e.g., a virus, a bacterium, or a fungus). For example, the invention can be used in a method of treating a viral infection by expressing an antisense RNA that hybridizes to an mRNA of an essential virus gene product (e.g., a polymerase mRNA). Other preferred antisense RNAs include, but are not limited to, those that are complementary to a naturally-occurring gene in the cell, which gene is expressed at an undesirably high level. For example, an antisense RNA can be designed to inhibit expression of an oncogene in a mammalian cell. Similarly, the virus vector of the present invention can be used to express a catalytic RNA (i.e., a ribozyme) that inhibits expression of a target gene in the cell by hydrolyzing an mRNA encoding the targeted gene product. Antisense RNAs and catalytic RNAs can be designed by employing conventional criteria.

If desired, the invention can be used to express a dominant negative mutant in a mammalian cell. For example, viral assembly in a cell can be inhibited or prevented by expressing in that cell a dominant negative mutant of a viral capsid protein. (see, e.g., Scaglioni et al., Virology 205:112-120, 1994; Scaglioni et al., Hepatology 24:1010-1017, 1996; Scaglioni et al., J. Virol. 71:345-353, 1997).

The invention can be used to express any of various “therapeutic” genes in a cell. A “therapeutic” gene is one that when expressed, confers a beneficial effect on the cell or tissue in which it is present, or on a mammal in which the gene is expressed. Examples of “beneficial effects” include amelioration of a sign or symptom of a condition or disease, prevention or inhibition of a condition or disease, or conferral of a desirable characteristic. Included among the therapeutic genes are those genes that correct a gene deficiency disorder in a cell or mammal. “Correction” of a gene deficiency disorder need not be equivalent to curing a patient suffering from a disorder. All that is required is conferral of a beneficial effect, including even temporary amelioration of signs or symptoms of the disorder. Also included are genes that are expressed in one cell, yet which confer a beneficial effect on a second cell. For example, a gene encoding a neurotransmitter can be expressed in a nerve cell, from which the neurotransmitter is then secreted to exert an effect on other nerve cells of the mammal. Other therapeutic genes include sequences that encode an antisense RNA that inhibits transcription or translation of a gene that is expressed at an undesirably high level. For example, an antisense gene that inhibits expression of a gene encoding an oncogenic protein is considered a therapeutic gene.

The invention can be used to express a therapeutic gene in order to treat a gene deficiency disorder. Particularly appropriate genes for expression include those genes that are expressed at a less than normal level in the target cells of the subject mammal. For example, in neurodegenerative disorders, such as, but not limited to, amyotrophic lateral sclerosis, primary lateral sclerosis, hereditary spastic hemiplegia, spinal muscular atrophy, bulbospinal atrophy, etc. a neuroprotective protein may be deficient, this neuroprotective protein will be supplied by the virus vector of the present invention.

Therapeutic and Prophylactic Utility

The method of the present invention can be used in one of many different species, including but not limited to, mammalian, bovine, ovine, porcine, equine, rodent and human. The invention features a method for treating a neurological disorder including, but not limited to, Cerebral Palsy, trauma induced paralysis, vascular ischaemia associated with stroke, neuronal tumours, motorneurone disease, Parkinson's disease, Huntington's disease, Alzheimer's disease, or multiple sclerosis. Further, peripheral neuropathies associated with diabetes, heavy metal or alcohol toxicity, renal failure and/or infectious diseases such as herpes, rubella, measles, chicken pox, HIV and/or HTLV-1, are also treated by the virus vector of the present invention. Disorders/conditions resulting from injuries to the central nervous system and/or any abnormality to the nervous system in a mammal will also benefit from the methods of the present invention.

The method involves (a) administering into a mammal a therapeutically effective amount of a non-mammalian retrovirus (e.g., a reticuloendotheliosis virus such as SNV) having a glycoprotein, the genome of which virus includes an exogenous gene encoding a therapeutic gene(s), (a protein, polypeptide or a sequence expressing a nucleic acid such as an antisense RNA), and (b) expressing the exogenous gene in the mammal. Particularly useful exogenous genes include, but are not limited to, those that encode therapeutic proteins such as nerve growth factor, hypoxanthine guanine phosphoribosyl transferase (HGPRT), tyrosine hydroxylase, dopadecarboxylase, brain-derived neurotrophic factor, basic fibroblast growth factor, glial derived neurotrophic factor (GDNF) and RETLI (also known as GDNFR.alpha., GFR-1, and TRN1). The invention further relates to the overexpression of genes that target various facets of nerve cell injury, including, but not limited to, those relating to calcium excess, accumulation of reactive oxygen species, protein malfolding, inflammation, and the induction of apoptosis. Further, expression of neuroprotective genes may be necessary in neurodegenerative conditions. Examples of neuroprotective genes include, but are not limited to, calbindin, bcl-2 and various growth factors.

Pharmaceutical Compositions

The present invention also provides pharmaceutical compositions. Such compositions comprise a therapeutically effective amount of the retroviral vector of the present invention expressing a therapeutic gene(s) and a pharmaceutically acceptable carrier or excipient. Such a carrier includes, but is not limited to, saline, buffered saline, dextrose, water, glycerol, ethanol, and combinations thereof. The carrier and composition can be sterile. The formulation should suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

### Results

In Vitro Studies.

To test the infectivity of retroviral vector particles pseudotyped with the envelope proteins of rabies viruses, transient transfection/infection experiments are performed (supra). 293T cells are transfected with plasmids expressing retroviral core proteins of a reticuloendotheliosis virus (plasmid pZP33), a plasmid containing a retroviral vector genome transducing the bacterial beta-galactosidase gene (plasmid pZP36), and various plasmids expressing the VSV G protein (positive control), or various rabies virus envelope proteins (strain B2C or N2C), or the spleen necrosis virus (SNV) envelope protein (SNV wt-Env). 48 hours after transfection, virus is harvested from transfected cell cultures and various dividing or growth-arrested target cells are infected.

Coinciding with previous results (Chu, T. H., et al., Gene. Ther. 1:292-299, 1994; Gautier, R., et al., J. Virol. 74:518-522, 1998), the retroviral particles containing the envelope protein of wild-type (wt) SNV do not infect human, mouse, or hamster cells (293T, NA, or BSR cells, respectively). However, they infected dog D17 cells (Table 1). Retroviral particles with the envelope protein of VSV infected all cell lines tested with vector titers up to 10⁴ colony forming units per ml tissue culture supernatant medium (cfu/ml) (Table 1). Particles pseudotyped with the rabies virus envelope protein of the virus strain B2C infected human, hamster, and mouse cells with titers similar to that obtained with particles pseudotyped with the envelope protein of VSV (Table 1). Retroviral vector particles pseudotyped with the envelope of the rabies virus strain N2C only infected dividing and growth-arrested neuronal cells (NA cells), but did not infect all other cell lines tested (Table 1).

In summary, in vitro experiments indicate that retroviral vector particles endowed with the envelope protein of rabies viruses are infectious in mammalian cells. Pseudotyping with the envelope protein of the neurotropic virus N2C allows for the specific transduction of neuronal cells.

TABLE 1
Infectivity of retroviral vector particles pseudotyped
with rabies virus envelope proteins

| | TARGET CELLS | | | | |
|---|---|---|---|---|---|
| pZP33 (gag-pal) + | | BSR (baby | NA (mouse | | D17 dog |
| pZP36 (lacZ) + | 293T | hamster kidney) | neuroblastoma) | | osteosarcoma |
| envelope | dividing | dividing | arrested | dividing | dividing |
| VSV-G | 5 × 10³ | 1 × 10⁴ | 1 × 10³ | nd | nd |
| B2C | 2 × 10³ | 2.7 × 10⁴ | nd | 2 × 10³ | nd |
| N2C | <10 | <10 | 3.7 × 10³ | 1.3 × 10⁴ | nd |
| SNV wt-Env | <10 | <10 | <10 | <10 | 7 × 10³ |

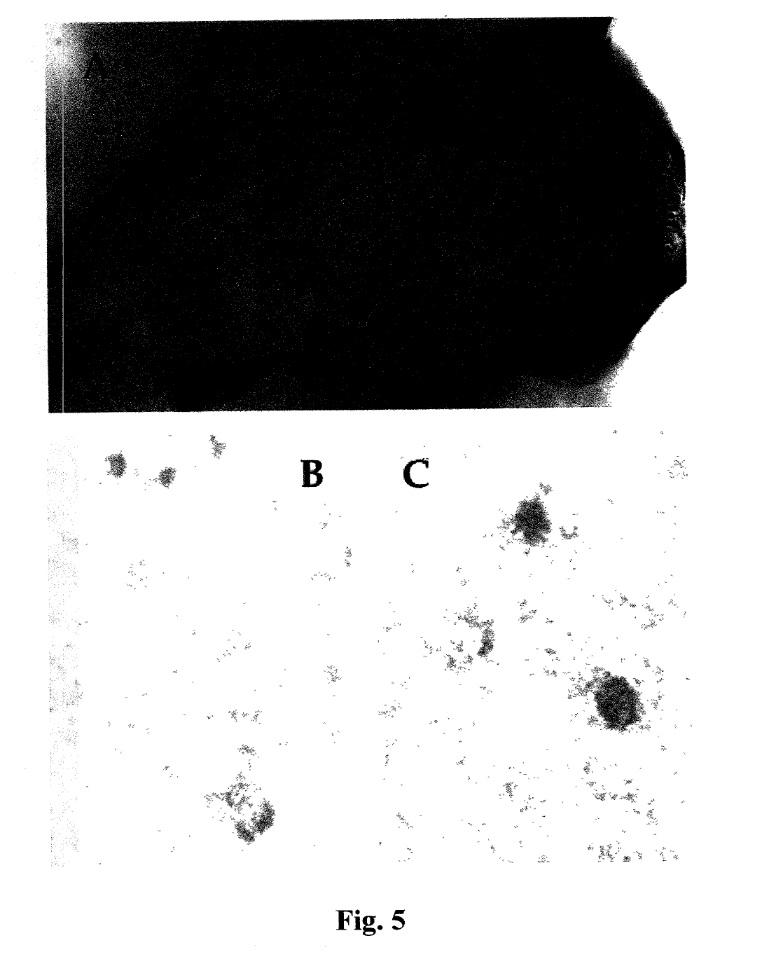

In vivo studies. To test the infectivity of retroviral particles pseudotyped with rabies virus envelope proteins, vector particles are injected into the brain of newborn mice (strain C57 BL/6 J-twi). 393T cells are transfected with plasmid pZP33, pZP36, and a plasmid expressing the rabies virus envelope protein of the strain N2C. 48 hours after transfection, virus is harvested and concentrated by ultracentrifugation (supra). 2 μl of concentrated virus solution (2×10⁵ infectious virus particles) are injected into the right lateral lobe or into the cerebellum. Two month after the injection the mice are sacrificed and the brain investigated for infected cells by X-gal staining (supra). Cells around the injection sites express the beta-galactosidase gene (FIG. 5). These data indicate that particles displaying the N2C envelope are infectious in viva as well as in vitro (supra).

What is claimed is:

1. A retroviral vector particle, comprising retroviral core proteins, a vector wherein the genome comprises an exogenous gene(s) of interest, a mammalian active promoter operably linked to said exogenous gene(s) of interest, and wherein said retroviral vector particle displays an envelope protein of a rabies virus.

2. The retroviral vector particle of claim 1, wherein said mammalian active promoter is a cell-type-specific promoter.

3. The retroviral vector particle of claim 1, wherein said mammalian active promoter is an inducible promoter.

4. The retroviral vector particle of claim 1, wherein said retroviral core proteins are derived from at least one of a spleen necrosis virus (SNV) or a reticuloendotheliosis virus (REV-A).

5. The retroviral vector particle of claim 1, wherein said envelope protein of a rabies virus is a glycoprotein of a rabies virus N2C strain.

6. A method for treating a disease or disorder in a mammal, comprising:
a) administering to a mammal a therapeutically effective amount of a retrovirus vector encoding an exogenous gene(s) of interest within a retrovirus vector particle, said retrovirus vector particle is pseudotyped with an envelope protein of a rabies virus;
b) transducing a target cell with said exogenous gene(s) of interest; and
c) expressing said exogenous gene(s) of interest in said mammal.

7. The method of claim 6, wherein said envelope protein of a rabies virus is an N2C glycoprotein.

8. The method of claim 6, wherein said target cell comprises a nerve cell.


SEQUENCE LISTING (2 sequences)

SEQ ID NO: 1 - DNA, 26 bp, Artificial Sequence
  PCR primers
cctctagaagatggttcctcaggctc

SEQ ID NO: 2 - DNA, 36 bp, Artificial Sequence
  PCR primers
tatagggcccaagctttcacagtctgatctcacctc